(19) United States
(12) Patent Application Publication
(10) Pub. No.: US 2022/0015928 A1
(43) Pub. Date: Jan. 20, 2022

(54) DAMPER SYSTEM

(71) Applicant: Ottobock SE & Co. KGAA, Duderstadt (DE)

(72) Inventors: Thorsten GROHS, Bovenden (DE); Andreas SCHUH, Duderstadt (DE); Michael Bremer, Göttingen (DE); Andreas KRUSE, Worbis (DE)

(73) Assignee: Ottobock SE & Co. KGAA, Duderstadt (DE)

(21) Appl. No.: 17/312,380

(22) PCT Filed: Dec. 10, 2019
(86) PCT No.: PCT/EP2019/084514

§ 371 Date: Jun. 9, 2021

(30) Foreign Application Priority Data
Dec. 11, 2018 (DE) 10 2018 131 698.4

(51) Int. Cl. A61F 2/60; A61F 2/68

(52) U.S. Cl. CPC A61F 2/60; A61F 2002/5006; A61F 2/68

(57) ABSTRACT

Damper system for orthopaedic devices, with a proximal fastening device and a distal fastening device which are coupled to each other in such a way as to be displaceable relative to each other, with a first hydraulic or pneumatic damper device, which is arranged between the fastening devices and has a flexion chamber and an extension chamber separated from each other by a movable piston and connected fluidically to each other by at least one overflow line, wherein a second damper device with a parallel action, and with a velocity-independent hysteresis behaviour, is arranged between the fastening devices and is configured as a tube structure damper.

The invention relates to a damper system for orthopedic devices, having a proximal fastening device and a distal fastening device which are coupled to each other and displaceable relative to each other, having a first hydraulic or pneumatic damper device, which is arranged between the fastening devices and has an extension chamber and a flexion chamber which are separated from each other by a movable piston and are fluidically connected to each other by at least one overflow line.

Pneumatic or hydraulic damper devices for orthopedic devices are used to influence relative movements between components of the orthopedic devices. Orthopedic devices are, for example, orthoses, exoskeletons or prostheses which are fastened to a patient. The orthopedic devices have components which are displaceable relative to one another, in particular pivotable or slidable, and whose relative movement can be influenced by a damper element. For example, pivoting movements are permitted by joints between an upper part and a lower part. These pivoting movements, which serve for extension and flexion of the joint, are influenced by hydraulic dampers or fluid dampers. Sliding movements between two components are likewise damped by a suitable arranged damper device. Hydraulic damping or pneumatic damping is generally effected by influencing the transport of fluid from a flexion chamber or extension chamber. In closed systems, the fluid is transported from the extension chamber into the flexion chamber and back. In pneumatic systems, damping can be effected by compression and/or controlled release from the respective chamber.

DE 10 2007 032 090 A1 discloses an orthopedic fluid damper which is designed for use in an orthosis or prosthesis. A displacement chamber, in which a piston is mounted, is formed in a cylinder housing. A fluid reservoir for a fluid is connected to the displacement chamber via a return flow line. A valve, which can assume an open position, and a closed position in which the return flow line is at least partially closed, is arranged in the return flow line.

EP 654254 A1 discloses a prosthetic joint with a joint upper part and a joint lower part, which are pivotably connected to each other. A damper device is integrated in one of the two joint parts and is designed as a rotary hydraulics system. A rotary piston divides a displacement chamber into two subchambers, which are connected to each other via two parallel, oppositely acting throttle check valves, wherein throttle points can be controlled separately from the outside in order to adjust or change the damping.

EP 672 398 B1 relates to a pivot connection between parts of an orthopedic aid with a multi-link, kinematic joint chain having at least four joint members, which are connected to one another via a common rotation axis. The rotation axes are oriented substantially parallel to one another. A joint member arranged on the flexion side is rotatably connected at its lower end, via an interposed joint connection, to a lower connecting joint member, wherein the interposed joint connection has two rotary connections on the flexion-side joint member and two rotary connections on the lower connecting joint member, such that, when the pivot connection moves, the joint member arranged on the flexion side executes a constrained movement relative to the lower connecting joint member, which comprises both a translation and a rotation. At least one resilient element can be arranged between two joint members; the resilient element can contain a damper.

EP 3 089 722 B1 relates to a prosthetic knee joint with an upper part, a lower part, and a four-part joint system, which is arranged on the upper part. The joint system is mounted on the lower part so as to be pivotable counter to a spring force during a stance phase flexion. A first spring element cushions the movement of the joint system with respect to the lower part about a pivot axis. A second spring element influences the movement within the joint system. The spring elements can each be designed as a spring-damper element or spring-damper system, wherein the damping can be made available via a fluid damper or an elastomer damper. The elastomer element can be designed as a tube or at least have a tube.

The object of the present invention is to make available a damper system for orthopedic devices, in particular for joint devices of the lower extremity, with which system it is possible to better adapt the damping behavior to the respective intended use and in particular to achieve a high level of reliability.

According to the invention, this object is achieved by a damper system having the features of the main claim. Advantageous embodiments and developments of the invention are disclosed in the subclaims, the description and the figures.

In the damper system according to the invention for orthopedic devices, having a proximal fastening device and a distal fastening device which are coupled to each other and displaceable relative to each other, having a first hydraulic or pneumatic damper device, which is arranged between the fastening devices and has an extension chamber and a flexion chamber which are separated from each other by a movable piston and are fluidically connected to each other by at least one overflow line, provision is made that a second damper device with parallel action, and with a speed-independent hysteresis behavior, is arranged between the fastening devices. While hydraulic or pneumatic damper devices have a speed-dependent hysteresis behavior, a parallel connection or a parallel arrangement of a second damper device makes it possible to combine different damping behaviors with one another and to make available different damping properties for different movement phases or load phases. For example, in the case of orthotic knee joints or prosthetic knee joints, it is possible to permit giving way or flexion in the stance phase. With a simultaneous damping effect of the two damper devices, different damper behaviors in the extension direction and in the flexion direction can result depending on the speed, such that different damper behaviors can be achieved and adjusted depending on the movement executed. In addition, if the hydraulic or pneumatic damper device fails, the further damper unit is always present, such that emergency operation with sufficient, albeit possibly no longer optimal, damper properties can still be ensured. The hydraulic damping or pneumatic damping is adjustable over a wide damping range and can be adapted to the respective patient or the intended use simply by reducing or increasing the flow cross section. The more powerful and more variable hydraulic or pneumatic damping has a higher probability of failure due to the movable components, which can be offset by the second damper device acting in parallel.

The second damper device can be constructed as a speed-independent damper, in particular as a friction-damping damper. However, an embodiment of the damper as a structure damper or elastomer damper can also have a hysteresis behavior that is substantially independent of the speed of deformation of the damper device. It is important that the energy use is substantially independent of the speed of deformation of the elastomer, such that another different damper characteristic can be made available with the second damper device. The deformation paths and thus the energy use at a constant spring rate are independent of the speed of deformation.

In a development of the invention, the second damper device is designed as a structure damper made from an elastomer, as a result of which, in addition to the damper effect of the second damper device, storage of force and a restoring force are also made available. The second damper device is designed as a combined spring-damper element, such that not only is the relative movement between the upper part and the lower part or the two fastening devices braked, but also a restoring force against giving way or further displacement of the components between which the damper system is arranged can be made available. The second damper device as a structure damper can be formed from a plastic composite, such that the second damper device is designed as an elastic solid body which has a constant and speed-independent reset behavior.

In a development of the invention, provision is made that the second damper device surrounds the first damper device circumferentially, whereby in the event of a leak, in particular of a hydraulic damper, it is possible to avoid a situation where hydraulic fluid escapes into the environment, or it is ensured that emerging hydraulic fluid is collected or at least stopped. The second damper device can completely surround and enclose the first damper device. The first damper device can be accommodated in the second damper device and can be secured thereto in a sealing or sealed manner, such that a module is obtained which ensures high durability and availability.

The damping behavior of the first damper device can be adjustable. The adjustment can be effected once for example, by adjusting valves or throttles, or else during the use of the damper system via actuators, such as motors or other actuating devices, which can be sensor-controlled or mechanical, for example, depending on movements or displacement situations of the fastening devices toward or away from each other. For example, depending on a measured joint angle or a direction of movement, a valve or a throttle can be opened or closed, or the respective flow cross section can be increased or decreased.

The second damper device can be designed as a tube damper or at least have a tube, it being possible to combine a plurality of tube elements with one another and to arrange them in series.

In a development of the invention, provision is made that the tube damper has at least one outwardly curved, barrel-shaped or bell-shaped portion, via the shape and design of which, for example in height, curvature or material thickness, it is possible to adjust the damping, the suspension behavior and the hysteresis behavior. By a suitable selection of the second damper element as a correspondingly designed and shaped tube damper with the respective barrel-shaped or bell-shaped portions, an adaptation to the respective intended use can be carried out to a large extent in cooperation with the first damper device. The tube damper can have a plurality of outwardly curved, barrel-shaped and/or bell-shaped portions with different diameters, material thicknesses and/or different heights, such that each tube portion has a different damper and/or spring characteristic, as a result of which it is possible to obtain a wide variation of spring and damper behavior.

The tube damper can have an inwardly and/or outwardly projecting support portion which bears on a housing, in particular on an outer face and/or inner face of a housing of the first damper device. By way of the inwardly and/or outwardly projecting support portion, it is possible to guide the tube damper on the outer face and/or inner wall of a housing of the first damper device. In addition, the first damper device is protected by a tube damper, which surrounds it on the outside, and permits a compact construction.

In a development of the invention, the first damper device is assigned a switching element, which at least reduces the flow resistance in the extension direction or flexion direction and keeps it at a set flow resistance in the opposite direction. This switching element can be used, for example, to permit a direction-dependent connection or disconnection of the first damper device. For example, if a lower resistance or a lower damping is required for the flexion of a joint device, the first damper device is switched off or its effect is reduced, such that, for example, the pneumatics or hydraulics are bridged. In the flexion direction, it is then only the second damper device or the structure damper made of an elastomer that acts, whereas in the reverse movement both the second damper device and the first damper device are activated in the form of the hydraulic or pneumatic damper device. The same can also take place the other way round, i.e. a double damper effect in the flexion direction and a single damper effect in the extension direction. This switchover can be dependent on the direction of movement and/or dependent on the load. The switching device can be designed, for example, as a changeover valve that is switched depending on the direction of flow.

The flow resistance of the first damper device can preferably be reduced in the compression direction of the second damper device.

In a development of the invention, provision is made that the hysteresis behavior of the first damper device is harder than the hysteresis behavior of the second damper device. The second damper device has a gentler hysteresis curve, that is to say a greater deviation between the damping behavior during the spring deflection compared to the damping behavior in the rebound.

A cylinder for receiving the piston of the first damper device can be formed in the fastening device in order to permit a compact construction of the damper system.

In a variant of the invention, provision is made that the second damper device is arranged within the medium of the first hydraulic or pneumatic damper device, i.e. within the fluid that is moved between the extension chamber and the flexion chamber during a displacement of the fastening devices relative to each other.

In a development of the invention, provision is made that at least one of the outwardly curved, barrel-shaped and/or bell-shaped portions, which can also have different diameters and/or different heights, is arranged above the first damper device, while at least one of the outwardly curved, barrel-shaped and/or bell-shaped portions is arranged below the first damper device. The tube structure damper is thus divided in two: one part above and one part below the first damper device.

Illustrative embodiments of the invention are explained in more detail below with reference to the attached figures, in which:

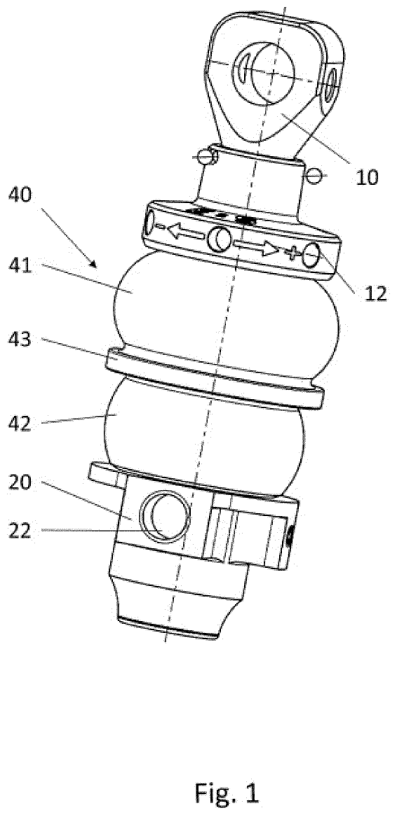

FIG. 1 shows an external view of an illustrative embodiment; and

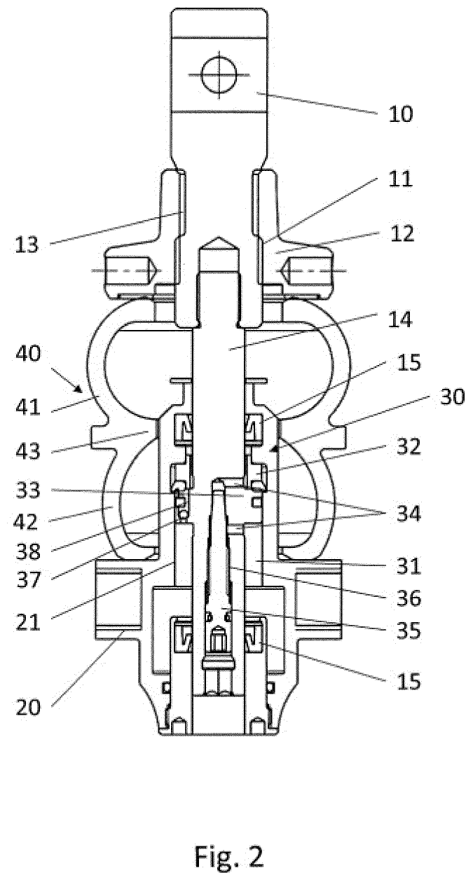

FIG. 2 shows a sectional view through a damper system according to FIG. 1;

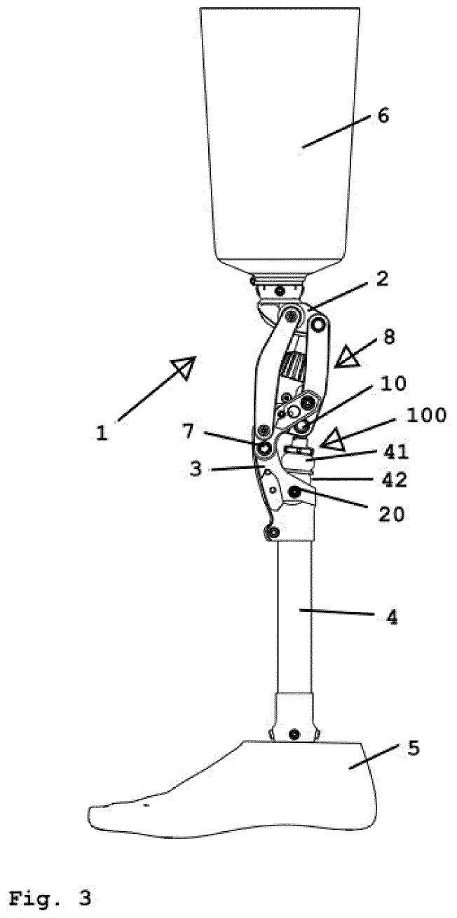

FIG. 3 shows a damper system in the installed state;

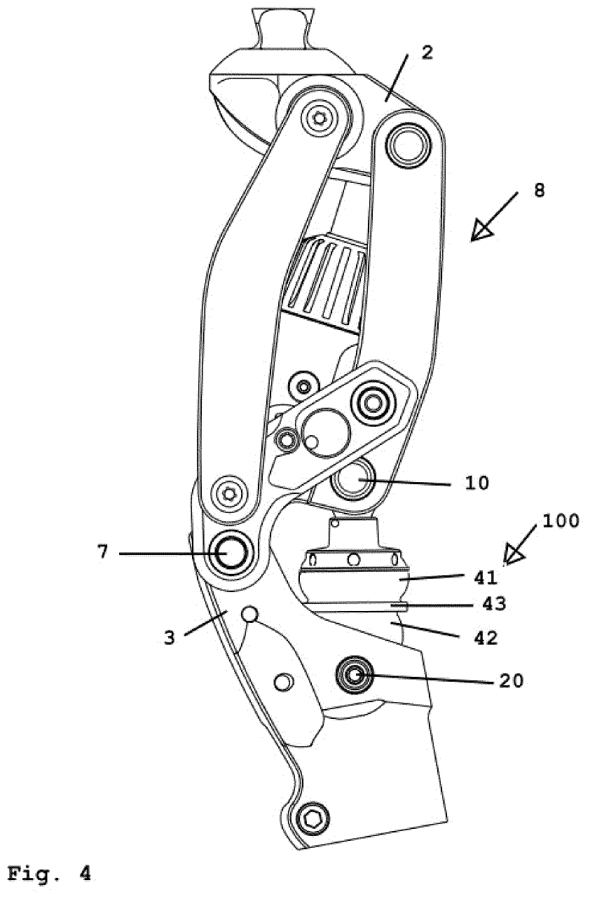

FIG. 4 shows a damper system in a prosthetic knee joint with stance phase flexion;

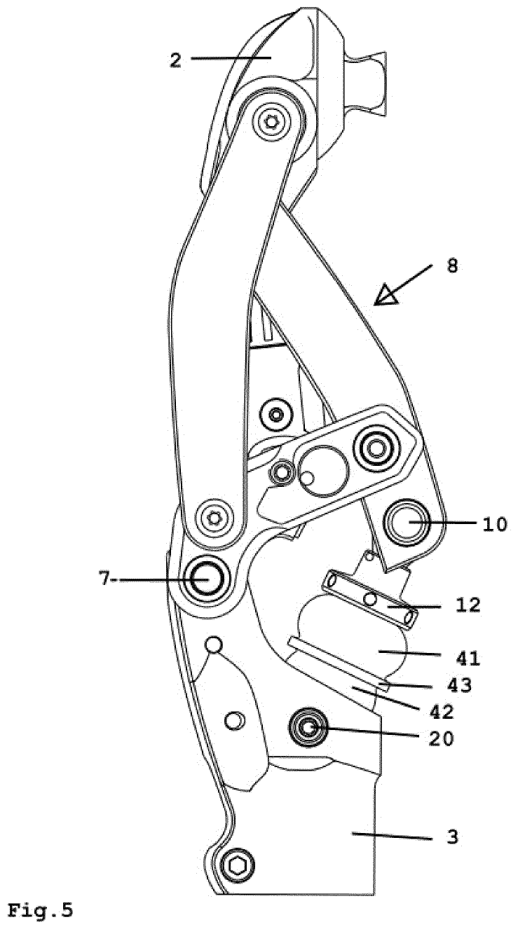

FIG. 5 shows a prosthetic knee joint according to FIG. 4 in a flexed state;

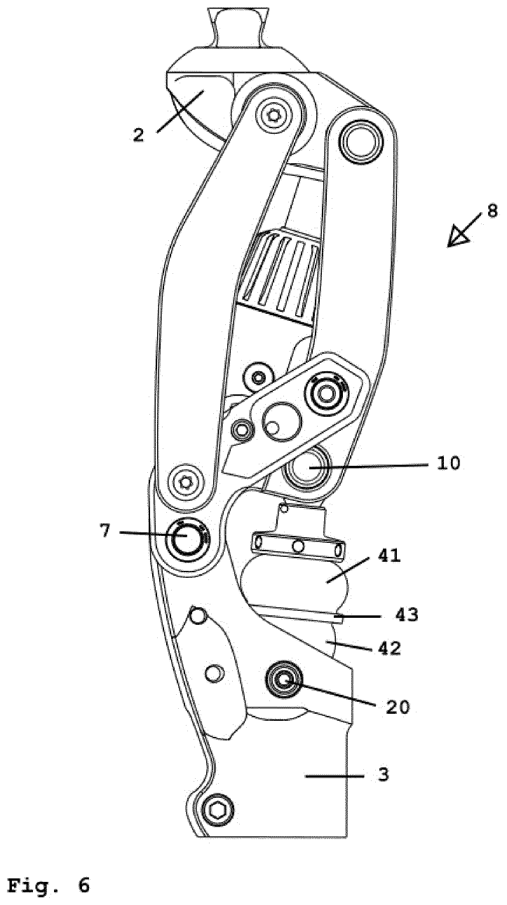

FIG. 6 shows the prosthetic knee joint according to FIG. 4 in an extended position;

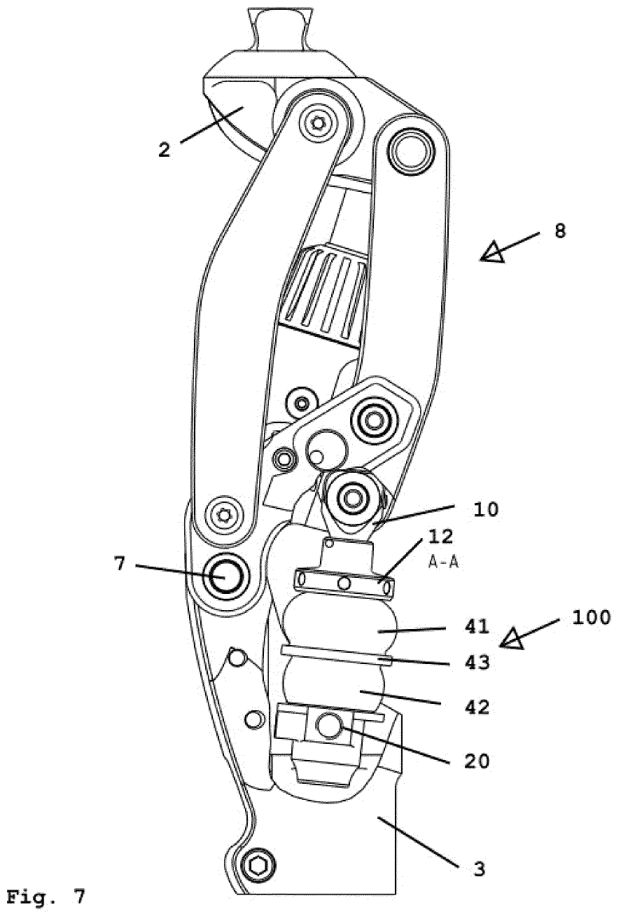

FIG. 7 shows the prosthetic knee joint according to FIG. 6 with a cut-open damper system;

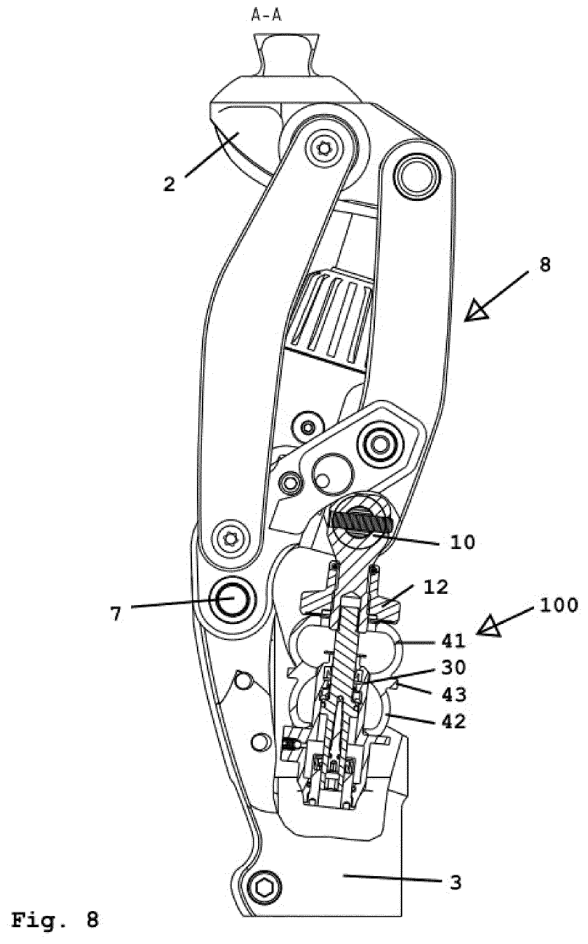

FIG. 8 shows FIG. 7 with the damper system in a sectional view;

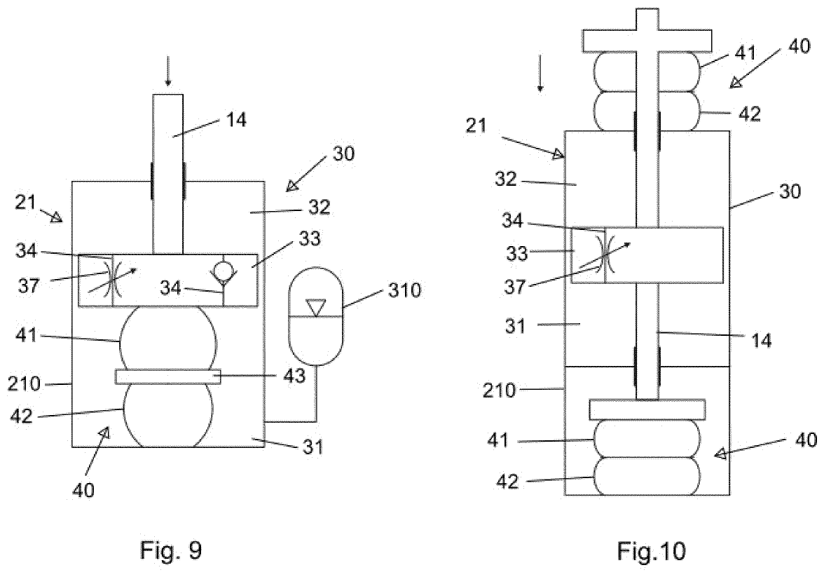

FIG. 9 shows a variant of the invention with a tube structure damper in the first damper device; and

FIG. 10 shows a variant of FIG. 9.

FIG. 1 shows a perspective view of a damper system for orthopedic devices, for example for prostheses, orthoses or exoskeletons, for damping relative movements between two components of the orthopedic device and, if necessary, for initiating restoring movements. The damper system has a proximal fastening device 10, via which the damper system can be secured to a component of the orthopedic device. The proximal fastening device 10 has a through-bore, through which a bolt or a screw can be guided in order to permit a reversible fastening and at the same time a pivotability about the bolt axis. On the proximal fastening device 10, an external thread is arranged or formed, in which an internal thread on a pretensioning element 12 engages. The pretensioning element 12 is mounted rotatably on the proximal fastening device 10 such that, by rotation in one or other direction, the pretensioning element is displaced along the longitudinal extent of the damper system. The pretensioning element 12 bears on an upper end of a second damper device 40, which is designed as a structure damper made from an elastomer. The second damper device 40 is designed as a tube damper with two bell-shaped or barrel-shaped portions 41, 42, which are arranged in a row one behind the other along the longitudinal extent of the damper system. Arranged between the two barrel-shaped, outwardly curved portions 41, 42 is a support portion 43, which is designed in the form of a disk. The further design of the second damper device 40 is explained with reference to FIG. 2.

The distal end of the second damper device 40 bears on a shoulder of a distal fastening device 20. In the illustrative embodiment shown, the distal fastening device 20 has two seats 22, each with an internal thread for receiving bolts or screws, so as to be able to secure the second or distal fastening device 20 to another component of the orthopedic device.

FIG. 2 shows a longitudinal sectional view of the damper system according to FIG. 1. The proximal fastening device 10 with the through-bore for receiving a bolt or a screw can be seen. A further bore, oriented perpendicular to the first through-bore, can be arranged in the bolt eye, for example in order to secure the proximal fastening device against rotation about the bolt axis.

Extending from the through-bore, along the longitudinal extent of the damper system, is a bolt on whose outer face the external thread 11 is formed which engages with an internal thread 13 of the pretensioning element 12. The pretensioning element 12 bears on the proximal, upper end of the second damper device 40 in the form of a tube damper. Within the bolt of the proximal fastening device 10, a piston rod 14 is screwed in. The piston rod 14 is guided movably in a cylinder 21, which is formed in the distal fastening device 20. On the piston rod 14, a piston 33 is arranged or formed which is movable to and fro in the cylinder 21 along the longitudinal extent of the damper system or of the piston rod 14. It is thereby possible for the proximal fastening device 10 to be moved in the direction of the distal fastening device 20 and back.

The distal fastening device 20 forms a housing in which the piston rod 14 is guided and which, at its proximal end, is sealed off by a seal 15. The seal 15 bears on the piston rod 14 and prevents escape of a hydraulic fluid which is arranged inside the cylinder 21 in an extension chamber 32 and a flexion chamber 31. The extension chamber 32 is separated from the flexion chamber 31 by the piston 33. Inside the piston rod 14, an overflow line is formed through which, upon displacement of the piston 33 due to a movement of the piston rod 14, hydraulic fluid can flow from the extension chamber 32 into the flexion chamber 31, when the two fastening devices 10, 20 are moved toward each other. When the movement is reversed, i.e. the fastening devices 10, 20 are moved away from each other, hydraulic fluid flows from the flexion chamber 31 through the overflow line 34 into the extension chamber 32. Within the overflow line 34, which in the illustrative embodiment shown passes through the piston rod 14 and the piston 33, a nozzle needle 35 is arranged, which is mounted adjustably on the outer face via a thread 36 in the piston rod 34. By turning the nozzle needle 35, it is possible to change the flow cross section of the overflow line 34. For this purpose, an access opening is formed at the distal end face of the distal fastening device 20, through which access opening a rotation of the nozzle needle can take place via a tool, for example a screwdriver, a polygon wrench or an Allen key. A further seal 15 for sealing off the piston rod 14 from the outside is arranged in the distal end region of the distal fastening device 20.

The piston rod 14 forms, together with the piston 33, the cylinder 21 and the overflow opening 34, a first, further damper device 30, which is designed as a hydraulic damper device 30. The first, hydraulic damper device 30 acts parallel to the second damper device 40, which is arranged between the pretensioning element 12 and the shoulder on the distal fastening device 20.

The second damper device 40 is designed as a tube damper made from an elastomer material and has two barrel-shaped, hollow portions 41, 42, which are connected in series to each other. Compared to the distal tube portion 42, the proximal tube portion 41 has a larger external diameter and a larger internal diameter and also a smaller longitudinal extent or height. The wall thickness of the two portions 41, 42 is substantially the same and constant, while the wall thicknesses increase in the proximal and distal end regions of the portions 41, 42. Between the two portions 41, 42, a solid support portion 43 is formed which has an external diameter corresponding substantially to the maximum external diameter of the proximal portion 41. The support portion 43 extends as far as the outer wall of the cylinder housing, which is configured by the distal fastening element 20. The support portion 43 provides radial guiding for the tube damper or the second damper element 40 when the two fastening devices 10, 20 are moved toward each other. In the event of a displacement of the fastening devices 10, 20 toward each other, not only is the piston 33 moved inside the cylinder 21, the two barrel-shaped portions 41, 42 are also compressed. On account of the hollow-body structure of the two portions 41, 42, they bulge outward, and the support portion 43 moves along a wall on the outer face of the cylinder 21 or along a wall of the housing for the hydraulic first damper device 30, downward in the direction of the distal fastening device 20. Thus, in addition to hydraulic damping on account of the impeded flow of fluid from the extension chamber 32 into the flexion chamber 31, a second, additional damping is made available via the tube damper 40 designed as a solid body. On account of the elastic configuration of the second damper device 30, this forms a spring-damper system which, in the absence of a force that moves the two fastening devices 10, 20 toward each other, initiates a restoring movement.

The first damper device 30 surrounds the first damper device 30 about the entire circumference and has a different damper behavior than the second damper device 40. In particular, the hysteresis behavior of the first damper device 30 is speed-dependent, while the second damper device 40 has a speed-independent hysteresis behavior. In addition, the first damper device 30 can be designed to be switchable and is adjustable over a wide range. By screwing in or unscrewing of the needle valve 35, it is possible to vary the flow cross section of the overflow line 34 over a wide range, as a result of which the damper properties of the hydraulic damper 30 can be changed accordingly. In addition, by means of a simple switching element 37 in the form of a check valve, it is possible for the hydraulic damper device 30 to be actively switched or bypassed depending on the direction of movement. In the case of a flexion movement, when the two fastening devices 10, 20 are moved toward each other, there is a high pressure on the side of the piston 33 facing toward the extension chamber 32. The check valve opens and frees a bypass channel 38, such that hydraulic fluid can flow from the extension chamber 32 into the flexion chamber 31 independently of an obstruction of the flow cross section in the overflow line 34. Conversely, with the switching element 37 in the form of a spring-loaded ball for example, during an extension movement, that is to say a reversal and an increase in the distance between the two fastening devices 10, 20 from each other, the bypass line 38 is closed, such that hydraulic fluid can flow from the flexion chamber 31 into the extension chamber 32 only through the overflow channel 34. It is thus possible to activate different damper devices for different movements. By spring-loading of the switching element 37, it is also possible to release the bypass line 38 in a manner depending on the load.

With a reverse arrangement of the switching element 37, it is possible to damp a flexion movement only via the second damper device 40 or to reduce the damping effect of the first, hydraulic damper device 30, whereas, in the reverse movement, both damper devices 30, 40 are fully activated. In addition to mechanical triggering, as is described in the illustrative embodiment, electronic triggering of the switching process can also take place.

FIG. 3 shows the damper system 100 in an installed state. The damper system 100 is part of a prosthesis 1 in the form of a prosthetic leg with a prosthetic knee joint, which has an upper part 2 with a proximally arranged thigh socket 6 thereon for receiving a thigh stump. The upper part 2 is articulated on a lower part 3 of the prosthetic knee joint. A lower-leg tube 4, with a prosthetic foot 5 arranged thereon, is fastened distally on the lower part 3. The upper part 2 is pivotable relative to the lower part 3 via a multi-link system 8, which is designed as a four-link system. The multi-link system 3 is additionally mounted on the lower part 3 about a separate pivot axis 7. About the pivot axis 7, which is arranged frontally and distally with respect to the multi-link system 8, the entire multi-link system 8, along with the upper part 2 and the thigh socket 6, can pivot counter to the direction of walking, i.e. clockwise in the illustrative embodiment shown. The multi-link system 8 and the upper part 2 are supported via the damper system 100 against pivoting about the pivot axis 7. For this purpose, the proximal fastening device 10 is fastened to the distal end of the posterior link of the multi-link system 8. The distal fastening device 20 is fastened to the lower part 3 such that, during pivoting about the pivot axis 7 in a stance phase flexion, the entire multi-link system 8, along with the upper part 2 and the thigh socket 6, pivots clockwise about the pivot axis 7, without the multi-link system 8 with the four joint axes performing a relative movement thereof to each other. In FIG. 3, the prosthesis 1 is shown in a maximally extended, unloaded state.

FIG. 4 shows only the prosthetic knee joint without the thigh socket 6 or lower-leg tube 4, with the upper part 2, the lower part 3, the multi-link system 8 and the pivot axis 7. In FIG. 3, the prosthetic knee joint is shown during a stance phase flexion, in which the prosthetic knee joint is not flexed with the multi-link system 8 but instead pivots about the pivot axis 7 in a clockwise direction under an axial load. Thus, when a load is placed on the heel or during flexion in the stance phase, the user or the patient subsides on account of the flexible damper system 100, for example in so-called heel strike, so that there is no undamped force introduced through the extended prosthetic leg into the stump or into the hip joint. The proximal and distal barrel-shaped or bell-shaped portions 41, 42 are compressed, by the stance phase load and by the movement of the proximal and distal fastening devices 10, 20 toward each other, and bulge outward. The support portion 43 divides the two portions 41, 42.

FIG. 5 shows the prosthetic knee joint according to FIGS. 3 and 4 in a flexed state. The upper part 2 is pivoted through 90° to the lower part 3, compared to the extended position according to FIG. 3. It will be seen that the multi-link system 8 has effected a displacement of the respective links relative to each other. Stance phase loading no longer takes place, the damper system 100 is relaxed, and the barrel-shaped or bell-shaped portions 41, 42 have returned to their original form.

FIG. 6 shows the prosthetic knee joint according to FIG. 3 in an enlarged detail. The prosthetic knee joint is fully extended, there is no stance phase load, the damper system 100 is accordingly not loaded, and the two fastening devices 10, 20 are at a maximum distance from each other.

FIG. 7 shows the damper system, in the position according to FIG. 6, in the installed state. Parts of the housing of the lower part 3 are cut away in order to illustrate the damper system 100, which corresponds substantially to the structure according to FIGS. 1 and 2.

FIG. 8 shows the damper system 100 according to FIG. 7 in a sectional view. The damper system 100 corresponds to the structure according to FIG. 2.

As an alternative to the first damper device 30 being encased by the second damper device 40 in the form of the tube structure damper, there is the possibility that a first portion 41 can be arranged above the first damper device 30 or proximally to the first damper device 30, and a second portion 42 can be arranged below or distally to the first damper device 30, such that the first damper device 30 can be embedded in series between two tube-structure damper portions or tube structure dampers 40. The proximally and distally arranged tube structure dampers 40, portions or second damper devices can have a plurality of outwardly curved, barrel-shaped and/or bell-shaped portions with different diameters and/or different heights.

FIG. 9 shows a schematic view of a damper system having a first hydraulic or pneumatic damper device 30 with a cylinder 21, which is surrounded by a cylinder wall in a cylinder housing 210. Inside the cylinder housing 210, the piston 33 of the first damper device 30 is arranged longitudinally displaceably on a piston rod 14. A fluid, for example a hydraulic fluid, is arranged both in the flexion chamber 31 and in the extension chamber 32. In the illustrative embodiment shown, the second damper device 40 with the two bell-shaped portions 41, 42 of the tube structure damper is arranged in the flexion chamber 31. The second damper device 40 bears on the side of the piston 33 facing away from the piston rod 14 and is compressed when the piston rod 14 together with the piston 33, for example during flexion of an orthopedic device, is displaced in the direction of the flexion chamber 31. This may be the case, for example, during the flexion of a joint. In order to compensate for the volume of the piston rod 14 entering the extension chamber 32, an expansion vessel 310 is assigned to the flexion chamber 31. When a compressive force is exerted on the piston rod 14, as is indicated by the arrow, for example during flexion of an orthotic or prosthetic joint, the piston 33 moves downward and compresses the first damper device 40. At the same time, hydraulic fluid or pneumatic fluid is forced from the flexion chamber into the expansion volume 310. Arranged inside the piston 33 are overflow lines 34, which are coupled to switching elements 37. A switching element 37 is designed as an adjustable valve or throttle in order to reduce the flow resistance through the overflow line 34 from the flexion chamber 31 into the extension chamber 32. Likewise, a check valve is arranged in a second overflow line 34 in order to make available a substantially unimpeded flow of the hydraulic fluid from the flexion chamber 31 into the extension chamber 32. The check valve can be spring loaded, such that it opens and frees the passage only when a limit pressure is reached.

During a reversal of the movement, i.e. during an extension of the joint and an outward excursion of the piston rod 14 from the extension chamber 32, the fluid flows out of the extension chamber 32 through the adjusting valve 37 through the overflow line 34 into the flexion chamber 32. In addition, volume compensation is effected by the fluid from the expansion tank 310.

In the illustrative embodiment according to FIG. 9, the second damper device 40 is completely embedded in the fluid within the first damper device 30. The support portion 43 can bear on the housing 210 or the cylinder wall of the hydraulic damper or pneumatic damper of the first damper device 30. To allow the fluid to flow into the expansion tank 310, at least one flow channel can be arranged in the support portion 43.

A variant of the invention is shown in FIG. 10, in which the piston 33 of the first damper device 30 is likewise arranged longitudinally displaceably in a fluid-filled cylinder 21 inside a housing 210. An overflow channel 34 is formed in the piston 33 and has a valve as a switching element 33, in order to at least reduce the flow resistance in the extension direction and/or flexion direction and, if necessary, to permit different settings for the extension direction and/or flexion direction.

In the illustrative embodiment shown in FIG. 10, two damper devices 40 are combined with the first damper device 30. Arranged at the upper end of the housing 210 is a first second damper device 40, which is arranged around the piston rod 14. During a flexion movement and an inward pressing of the piston 33 in the direction of the flexion chamber 31, the upper second damper device 40 is compressed. Likewise, a lower second damper device 40, located in a chamber inside the housing 210 separated from the flexion chamber 31, is compressed in order to damp the flexion movement. An expansion tank is not necessary since, on account of the continuous piston rod 14, there are no volume fluctuations to be compensated. The lower second damper device 40 can be located in a hydraulic fluid; it is likewise possible that the chamber below the flexion chamber 31 is otherwise empty or filled with the ambient air that is in exchange with the ambient air.

During an extension movement, i.e. an upward movement of the piston 33 inside the housing 210, the elements of the tube structure dampers of the second damper devices 40 relax.

1. A damper system for orthopedic devices, having a proximal fastening device and a distal fastening device which are coupled to each other and displaceable relative to each other, having a first hydraulic or pneumatic damper device, which is arranged between the proximal and distal fastening devices and which has a flexion chamber and an extension chamber which are separated from each other by a movable piston and which are fluidically connected to each other by at least one overflow line, the damper system further including a second damper device with parallel action, and with a speed-independent hysteresis behavior, the second damper device being arranged between the proximal and distal-fastening devices and being designed as a tube structure damper.

2. The damper system of claim 1, wherein the second damper device is made from an elastomer.

3. The damper system of claim 1, wherein the second damper device surrounds the first damper device circumferentially.

4. The damper system of claim 1, wherein the damping behavior of the first damper device is adjustable.

5. The damper system of claim 1, wherein the tube structure damper has at least one outwardly curved, barrel-shaped or bell-shaped portion.

6. The damper system of claim 1, wherein the tube structure damper has a plurality of outwardly curved, barrel-shaped and/or bell-shaped portions with different diameters and/or different heights.

7. The damper system of claim 1, wherein the tube structure damper has an inwardly and/or outwardly projecting support portion, which bears on a housing of the first damper device.

8. The damper system of claim 1, wherein the first damper device is assigned a switching element which at least reduces the flow resistance in the extension direction or flexion direction and keeps it at a set flow resistance value in the opposite direction.

9. The damper system of claim 8, wherein the flow resistance of the first damper device is reduced in the compression direction of the second damper device.

10. The damper system of claim 1, wherein the hysteresis behavior of the second damper device is softer than that of the first damper device.

11. The damper system of claim 1, wherein one of the fastening devices forms a cylinder for receiving the piston.

12. The damper system of claim 1, wherein the second damper device is arranged within the fluid of the first damper device.

13. The damper system of claim 6, wherein at least one of the barrel-shaped and/or bell-shaped portions is arranged above the first damper device, and at least one of the barrel-shaped and/or bell-shaped portions is arranged below the first damper device.

14. A damper system for orthopedic devices, the damper system comprising:
a proximal fastening device and a distal fastening device, the proximal and distal fastening devices being coupled to each other and displaceable relative to each other;
a first hydraulic or pneumatic damper device arranged between the proximal and distal fastening devices, the first hydraulic or pneumatic damper device having a flexion chamber and an extension chamber which are separated from each other by a movable piston and which are fluidically connected to each other by at least one overflow line;
the damper system further including a second damper device configured as a tube structure damper device made from an elastomer which surrounds the first damper device circumferentially, the second damper device having parallel action and with a speed-independent hysteresis behavior, the second damper device being arranged between the proximal and distal fastening devices.

15. The damper system of claim 14, wherein the damping behavior of the first damper device is adjustable.

16. The damper system of claim 14, wherein the tube structure damper has at least one outwardly curved, barrel-shaped or bell-shaped portion.

17. The damper system of claim 16, wherein the tube structure damper has a plurality of outwardly curved, barrel-shaped and/or bell-shaped portions with different diameters and/or different heights.

18. The damper system of claim 1, wherein the tube structure damper has an inwardly and/or outwardly projecting support portion, which bears on a housing of the first damper device.

19. The damper system of claim 1, wherein the first damper device is assigned a switching element which at least reduces the flow resistance in the extension direction or flexion direction and keeps it at a set flow resistance value in the opposite direction.

20. A damper system for orthopedic devices, the damper system comprising:
a proximal fastening device and a distal fastening device, the proximal and distal fastening devices being coupled to each other and displaceable relative to each other;
a first hydraulic or pneumatic damper device with adjustable damping behavior, the first hydraulic or pneumatic damper device being arranged between the proximal and distal fastening devices, and the first hydraulic or pneumatic damper device having a flexion chamber and an extension chamber which are separated from each other by a movable piston and which are fluidically connected to each other by at least one overflow line;
the damper system further including a second damper device configured as a tube structure damper device with at least one outwardly curved, barrel-shaped or bell-shaped portion, the tube structure damper device being made from an elastomer and which surrounds the first damper device circumferentially, the second damper device having parallel action and with a speed-independent hysteresis behavior, the second damper device being arranged between the proximal and distal fastening devices.
